(19) United States
(12) Patent Application Publication
(10) Pub. No.: US 2022/0187277 A1
(43) Pub. Date: Jun. 16, 2022

(54) DEVICE FOR MEASURING HEAVY METALS HAVING ELECTRODE ELEMENT

(71) Applicants: Yonsei University Industry Foundation, Seoul (KR); Nformare Inc., Seoul (KR)

(72) Inventors: Heon Jin CHOI, Seoul (KR); Jae Suk SUNG, Seoul (KR); Youngcheol CHAE, Seoul (KR)

(73) Assignees: Yonsei University Industry Foundation, Seoul (KR); Nformare Inc., Seoul (KR)

(21) Appl. No.: 17/124,394

(22) Filed: Dec. 16, 2020

(51) Int. Cl. G01N 33/487

(52) U.S. Cl. CPC G01N 33/48714; A01K 61/95; G01N 33/48785

(57) ABSTRACT

Provided herein is a device for measuring heavy metals having an electrode element and, most particularly, a device for measuring heavy metals having an electrode element including a device for measuring heavy metals having an electrode element including a substrate having a wireless communication electrode for wirelessly delivering an electrical variance that is measured from an electrode, when measuring heavy metals, an electrode element delivering an electrical signal for measuring heavy metals, and a controller controlling electric current application to the electrode element and detecting a quantity of electricity between electrodes being measured from a measurement object contaminated by heavy metal, or calculating a displacement between a reference quantity of electricity and a measured quantity of electricity.

### TECHNICAL FIELD

The present invention relates to a device for measuring heavy metals having an electrode element and, most particularly, to a device for measuring heavy metals having an electrode element that enables heavy metals to be directly (or personally) measured from liquids as well as solid food, unlike the conventional method of performing detection of heavy metals using general chemical reactions, by using an invasive electrode element for a measurement object and by applying an electrochemical technology for precisely measuring impedance change between electrodes, change in phase difference, and so on.

### BACKGROUND ART

In numerous countries and, most particularly, in developing countries around the world, people are living in unsanitary living environments due to critical environmental issues and are exposed to a wide range of problems resulting from environmental pollution. Such pollution occurs in various forms, and, herein, heavy metal pollution (or contamination) is widespread throughout the atmosphere (or air), water, soil, and so on. Most particularly, heavy metals that are accumulated in the soil are transferred to food, such as agricultural produce, fish, and so on. Thus, the heavy metals are passed on to people, i.e., the end users, thereby causing various types of diseases resulting from heavy metal poisoning.

Additionally, developing countries are building more factories in order to quickly achieve industrial growth. Most of the factories are related to factories processing leather, dyeing factories, plating factories, and so on, which are generally avoided in many of the advanced and developed countries. Moreover, since raw materials, base materials, chemicals, and so on, that are used during the process of manufacturing products related to the factories listed above, workers working in the industrial sites are constantly exposed to the dangers of heavy metal poisoning. Furthermore, problems caused by heavy metals are also occurring near mining waste sites. Herein, abandoned and dead mines, mine water, and so on, which are generated during the mining, separation, and refining processes that are performed in metal mines, have been identified as the cause of such problems.

The most typical pollution related diseases caused by heavy metal pollution (or contamination) are the “Minamata disease”, which is caused by Mercury poisoning, and the “Itai-itai disease”, which is caused by Cadmium poisoning. More specifically, the “Minamata disease” is known to be caused by food made of marine plants that are contaminated with Mercury, and the “Itai-itai disease” is known to be caused by a long-term consumption of rice that is contaminated with Cadmium. Accordingly, poisoning caused by heavy metal pollution is being recognized as a critical problem.

Although the essential measures for preventing diseases related to heavy metal poisoning is to resolve the environmental problems so that heavy metal pollution can no longer occur. However, since this corresponds to a national problem-solving method that shall take a considerably long period of time, avoiding the consumption of drinking water or food containing heavy metals shall be one of the first realistic prevention methods. However, according to the current technology, highly expensive special equipment is needed to detect the presence of heavy metals, and experts and specialists are needed to analyze whether or not heavy metals are present. Therefore, whether or not a particular type of food contains heavy metals cannot be immediately verified (or confirmed) when the food is to be consumed. Additionally, since such detection is recognized as a professional field, and since the number of experts and specialists and the number of required equipment is limited, there are many difficulties in gaining regional and local detailed data of a vast region (or area) within a short period of time.

Currently, the commercially used detection device for detecting heavy metals is a small-sized heavy metal detection kit, which is an instrument that is used for measuring levels of pollution in water, such as measuring water quality. That is, this instrument (or kit) uses a method of placing a test strip in water that needs to be measured (or tested) and then observing the test strip for a change in color. In this method, there lies a problem in that the object of measurement (or measurement object) needs to be in no other form than liquid, such as water. Therefore, this method cannot be used on solid food, which is generally consumed by people. Furthermore, in order to perform heavy metal detection on solid food, liquid needs to be separated and extracted from the solid food by using a highly professional method, which then requires the usage of high-performance equipment for analysis. Thus, as described above, there are limitations in carrying out such tests.

### DETAILED DESCRIPTION OF THE INVENTION

### Technical Objects

The present invention is devised to resolve the above-described technical problems of the related art, and, therefore, a technical object of the present invention is to use an invasive electrode element so as to allow measurement of heavy metals to be performed on measurement objects that are in liquid state as well as in solid state.

Additionally, another object of the present invention is to allow any person to perform heavy metal measurement actions, even if the person is not a professional or specialist in heavy metal measurement.

Additionally, another object of the present invention is to enhance precision in the heavy metal measurement by using a technology that can precisely measure changes in the quantity of electricity between electrodes, e.g., changes in impedance or capacitance, and so on.

Additionally, due to its compact size and portability, another object of the present invention is to enable heavy metal measurement to be performed in real time at any location.

Additionally, since the present invention allows any person to easily measure heavy metal in a vast region (or area), another object of the present invention is to enable, for example, a heavy metal distribution map to be produced by collecting and processing heavy metal current status data corresponding to various regions within a shorter period of time, as compared to the method involving experts and specialists and using highly expensive equipment.

Additionally, another object of the present invention is to allow various of types of heavy metals to be detected by using a single device (or instrument) that is configured to be capable of changing electrode types.

Additionally, another object of the present invention is to allow multiple types of heavy metals to be detected simultaneously by using a single device (or instrument) by operating different types of electrode parts at the same.

Additionally, another object of the present invention is to allow the heavy metal distribution map to be updated in real time by equipping the device for measuring heavy metals according to the present invention with a wireless communication function, so as to enable the device to directly and automatically store the measured heavy metal current status data to a cloud or to primarily transmit the data to a neighboring smartphone and then to automatically store the primarily transmitted data to a cloud by using the smartphone.

Furthermore, since there is no limitation in the properties and shapes of the test object(s), and since the presence or absence of heavy metal contamination can be verified by a simple process, another object of the present invention is to immediately determine whether or not the food that is to be consumed contains heavy metals even by using a non-professional method.

### Technical Solutions

In order to achieve the above-described objects of the present invention, provided herein is a device for measuring heavy metals having an electrode element including a substrate having a wireless communication electrode for wirelessly delivering an electrical variance that is measured from an electrode, when measuring heavy metals, an electrode element delivering an electrical signal for measuring heavy metals, and a controller controlling electric current application to the electrode element and detecting a quantity of electricity between electrodes being measured from a measurement object contaminated by heavy metal, or calculating a displacement between a reference quantity of electricity and a measured quantity of electricity.

It is preferable that the electrode element includes a base substrate, and at least one or more pillar-type electrode parts protruding from the base substrate, or a flat-type electrode part.

It is preferable that the electrode part is coated with a receptor reacting to each heavy metal.

It is preferable that the electrode part is configured of multiple microelectrodes, that counter electrode groups are configured of one microelectrode being a reference electrode and other microelectrodes each being positioned at different positions at a same distance from the reference electrode, and that capacitance or impedance measurement values are obtained between the reference electrode and each microelectrode of the counter electrode groups.

It is preferable that an average value of measurement values for each counter electrode group is obtained, or that an average value of the measurement values excluding at least one of a highest value or a lowest value, among the measurement values for each counter electrode group, is obtained.

It is preferable that multiple microelectrodes configure a pattern of a same number of microelectrodes or different numbers of microelectrodes being aligned along a longitudinal direction and a transverse direction.

It is preferable that multiple microelectrodes are positioned at a center of multiple concentric circles and aligned along a circumference of each concentric circle, that a reference electrode is a microelectrode being positioned at the center, and that the counter electrode group is a group of microelectrodes being aligned along the circumference of each concentric circle.

It is preferable that multiple microelectrodes being aligned on the substrate are divided into a predetermined plurality of sections, and that the microelectrodes of each section are configured to detect substances that are different from one another.

Additionally, provided herein is a method for measuring heavy metals and performing update in real time including the steps of transmitting measured heavy metal data by a wireless communication electrode, thereby allowing heavy metal data to be stored in a cloud or allowing heavy metal data to be stored in a cloud via a portable terminal, sorting the data per region, and marking heavy metal distribution on a map based on the data being sorted per region, wherein the heavy metal distribution is updated in real time when updating the heavy metal data.

It is preferable that the method includes the steps of counting a number of measurements of the heavy metal data, performing calculation to convert the measurement numbers to a monetary reward for each measurer, and providing the monetary reward to each measurer.

### Effects of the Invention

According to the present invention, expected herein is an effect of allowing measurement of heavy metals to be performed on measurement objects that are in liquid state as well as in solid state.

Additionally, according to the present invention, expected herein is an effect of allowing any person to perform heavy metal measurement actions, even if the person is not a professional or specialist in heavy metal measurement.

Additionally, according to the present invention, expected herein is an effect of enhancing precision in the heavy metal measurement by using a technology that can precisely measure changes in the quantity of electricity between electrodes, e.g., changes in impedance or capacitance, and so on.

Additionally, according to the present invention, due to its compact size and portability, expected herein is an effect of enabling heavy metal measurement to be performed in real time at any location.

Additionally, since the present invention allows any person to easily measure heavy metal in a vast region (or area), expected herein is an effect of enabling, for example, a heavy metal distribution map to be produced by collecting and processing heavy metal current status data corresponding to various regions within a shorter period of time, as compared to the method involving experts and specialists and using highly expensive equipment.

Additionally, according to the present invention, expected herein is an effect of allowing various of types of heavy metals to be detected by using a single device (or instrument) that is configured to be capable of changing electrode types.

Additionally, according to the present invention, expected herein is an effect of allowing multiple types of heavy metals to be detected simultaneously by using a single device (or instrument) by operating different types of electrode parts at the same.

Additionally, according to the present invention, expected herein is an effect of allowing the heavy metal distribution map to be updated in real time by equipping the device for measuring heavy metals according to the present invention with a wireless communication function, so as to enable the device to directly and automatically store the measured heavy metal current status data to a cloud or to primarily transmit the data to a neighboring smartphone and then to automatically store the primarily transmitted data to a cloud by using the smartphone.

Furthermore, according to the present invention, since there is no limitation in the properties and shapes of the test object(s), and since the presence or absence of heavy metal contamination can be verified by a simple process, expected herein is an effect of immediately determining whether or not the food that is to be consumed contains heavy metals even by using a non-professional method.

### BRIEF DESCRIPTION OF THE DRAWINGS

FIG. 1 is a mimetic diagram showing a state of using a heavy metal measuring device according to an embodiment of the present invention in order to measure heavy metals in fish.

FIG. 2 is a mimetic diagram for describing a measurement principle of a heavy metal measuring device according to an embodiment of the present invention.

FIG. 3 is a mimetic diagram for describing changes in quantity of electricity that are shown according to heavy metal contamination states, when applying the heavy metal measuring device according to an embodiment of the present invention to fish, which is the given object of measurement.

FIG. 4 is a graph showing quantities of electricity according to different types of heavy metals when performing heavy metal measurement using the heavy metal measuring device according to an embodiment of the present invention.

FIG. 5 is a flowchart showing a process of collecting big data, from the heavy metal measuring device according to an embodiment of the present invention, and completing a platform for heavy metal measurement.

FIG. 6 is a flowchart for describing a process of collecting data by using the heavy metal measuring device according to an embodiment of the present invention.

FIG. 7 is a mimetic diagram of the heavy metal measuring device having an electrode array pattern according to an embodiment of the present invention.

FIG. 8 to FIG. 10 show various embodiments of an electrode array pattern according to the present invention.

FIG. 11 shows comparison data in results from measuring properties by using the method proposed in the present invention.

FIG. 12 shows an embodiment of an array pattern being divided into multiple sections according to an embodiment of the present invention.

### MODE FOR CARRYING OUT THE INVENTION

Hereinafter, in order to allow anyone with ordinary knowledge and skills in the art to easily carry out the present invention, exemplary embodiments of the present invention will be described in detail with reference to the appended drawings. However, the present invention may be implemented in various forms and shall not be limited only to the exemplary embodiments described herein. Additionally, in the appended drawings, for clarity in the description of the present invention, parts that are not related to the description of the present invention have been omitted from the drawings, and, throughout the entire specification, similar parts have been assigned with similar reference numerals.

Throughout the entire specification, it shall be understood that, when a particular part is said to “include” a particular component, unless specified otherwise, this means that other components may be further included and does not mean that other components are excluded.

Furthermore, terms such as “ . . . unit”, “ . . . part”, and so on, means a unit performing or processing at least one function or operation.

In the present invention, although food and drinks are mainly given as test objects for detecting heavy metals, the test objects will not be limited, as long as the test object is/has a region (or area) to which the device for measuring heavy metals (100) (or heavy metal measuring device (100)) can be equipped (or installed).

The heavy metal measuring device (100) according to the present invention is configured as described below.

Firstly, a substrate (103) having a wireless communication electrode for wirelessly delivering electrical variance (or change) that is measured from an electrode, when measuring heavy metals.

The wireless communication electrode is an electrode, e.g., antenna, for delivering data that is to be transmitted from a controller (101) to a server in a signal form.

Secondly, an electrode element delivering an electrical signal for measuring heavy metals may be configured, wherein the electrode element may be configured of multiple electrode parts (107) (which may also be shortened to “electrode”). And, herein, when power is supplied, a change in the quantity of electricity according to a medium, which exists between two electrode parts (107) is measured. This is the basic principle of heavy metal measurement.

Thirdly, a controller (101) for controlling electric current application to the electrode element and for detecting a quantity of electricity between electrode parts (107) being measured from a contaminated measurement object, or for calculating a displacement between a reference quantity of electricity and a measured quantity of electricity. The controller (101) may include a calculation module for performing calculation, and the calculation module may be mounted on a separate server (e.g., cloud or edge cloud server, and so on).

Herein, the electrode element includes a base substrate (105), and at least one or more pillar-type electrode parts (107) protruding from the base substrate (105), or a flat-type electrode part (107).

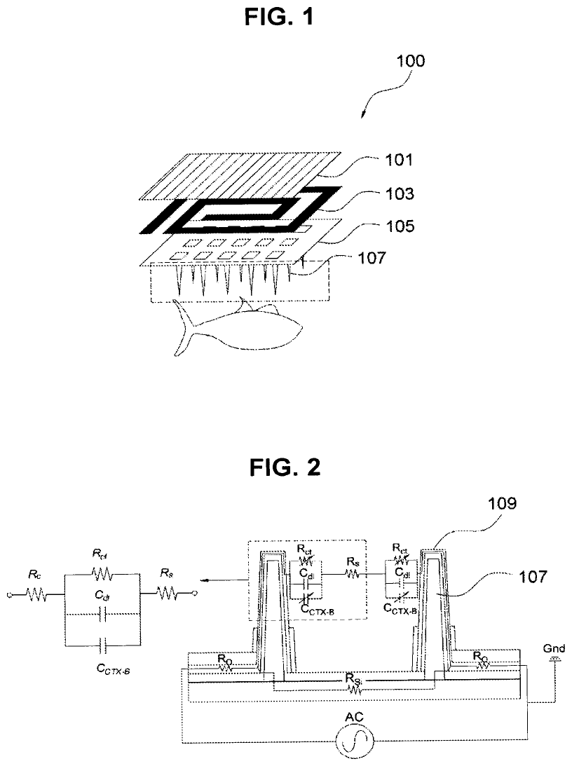

FIG. 1 is a mimetic diagram showing a state of using a heavy metal measuring device (100) according to an embodiment of the present invention in order to measure heavy metals in fish.

As shown in the drawing, by invading (or inserting) an electrode part (107) directly to solid food, such as fish, the heavy metal measuring device (100) according to the present invention may easily and simply measure heavy metals inside the corresponding solid food. Since a change in the quantity of electricity between multiple electrodes, which is detected by the heavy metal measuring device (100), varies for each type of heavy metal, whether or not heavy metal is measured and a type of the heavy metal may be determined based on the corresponding change in the quantity of electricity. The heavy metal measuring device (100) according to the present invention transmits the changed quantity of electricity to a server or terminal (or user equipment) through a means of communication and, then, analyzes the transmitted information from the server or terminal (or user equipment), or, by having the controller (101) be equipped with a calculation module, the heavy metal measuring device (100) may determine whether or not heavy metals exist and the type of heavy metal based on changes in the quantity of electricity between electrodes.

FIG. 2 is a mimetic diagram for describing a measurement principle of a heavy metal measuring device (100) according to an embodiment of the present invention. As shown in the drawing, the heavy metal measuring device (100) according to the present invention determines the presence or absence of heavy metals based on changes in impedance and capacitance between electrode parts (107). Herein, if heavy metals exist in the measurement object, properties of the measurement object matrix resulting from heavy metal ions changes, and, in comparison to when there are no heavy metals, the electrical properties, such as resistance value (impedance), or capacitance, and so on, according to a frequency between two electrodes may change minutely. Additionally, in order to enhance accuracy by maximizing such differences, which result from the presence or absence of heavy metals, and in order to enhance selectivity by verifying which type of heavy metal is present by differentiating the property values of each heavy metal, a receptor (109), such as an artificial peptide that is capable of separately reacting to each heavy metal, may be attached to the surface of each electrode in the form of a coated layer.

Herein, the change in the quantity of electricity denotes a change in quantity of electric current, impedance, capacitance, and so on, and the quantity of electricity includes all property values that may be measured between both ends of an electrode.

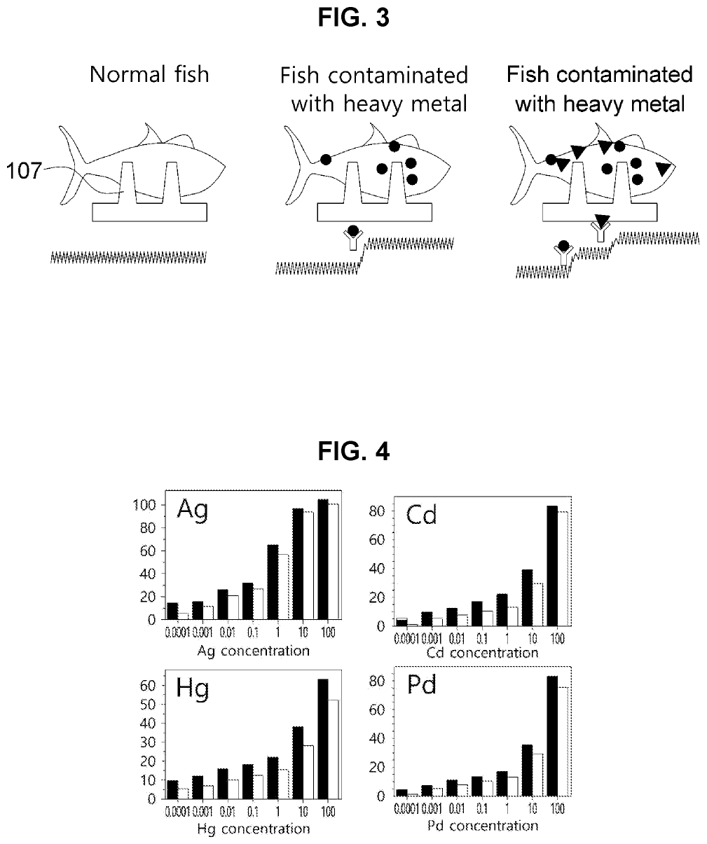

FIG. 3 is a mimetic diagram for describing changes in quantity of electricity between multiple adjacent electrode parts (107) that are shown according to heavy metal contamination states, when applying the heavy metal measuring device (100) according to an embodiment of the present invention to fish, which is the given object of measurement. As shown in the drawing, in case a heavy metal does not exist inside the body of a fish, for example, an impedance change does not exist between multiple electrode parts (107), and a constant impedance is measured. However, in case one type of heavy metal exists inside the body of a fish, a change in impedance occurs, and in case another type of heavy metal exists, another change in impedance occurs. Thus, the presence or absence of heavy metals and the types of heavy metals being present may be specified based on such change.

Herein, in case one heavy metal exists, only one type of impedance change occurs. And, in case two heavy metals exist, two types of impedance change occur. For example, an electrode part (107) dedicated to measuring Mercury may be inserted so as to measure the presence or absence of Mercury, and an electrode part (107) dedicated to measuring Cadmium may be inserted so as to measure the presence or absence of Cadmium.

Alternatively, an array of multiple electrode parts (107) may be divided into multiple sections, wherein one section may be assigned with a Mercury measuring electrode part (107), and wherein another section may be assigned with a Cadmium measuring electrode part (107).

This, for example, may also be implemented by coating a receptor (109), as described above. Evidently, heavy metal measurement may also be performed, without any receptor (109), by measuring a difference in the quantity of electricity occurring between multiple electrode parts (107).

FIG. 4 is a graph showing quantities of electricity according to different types of heavy metals when performing heavy metal measurement using the heavy metal measuring device (100) according to an embodiment of the present invention. As shown in the drawing, it is apparent that a rate-of-change in impedance changes in proportion to a concentration of heavy metal. Herein, the dark-shaded bar indicates a status value of an electrode part (107) being coated with a receptor (109), such as an artificial peptide, as described above, and the light-shaded bar indicates a status value of an electrode part (107) that is not coated with a receptor (109). Herein, in case the electrode part (107) is coated with a receptor (109), a larger rate-of-change in impedance is shown, which indicates that measurement sensitivity is slightly improved.

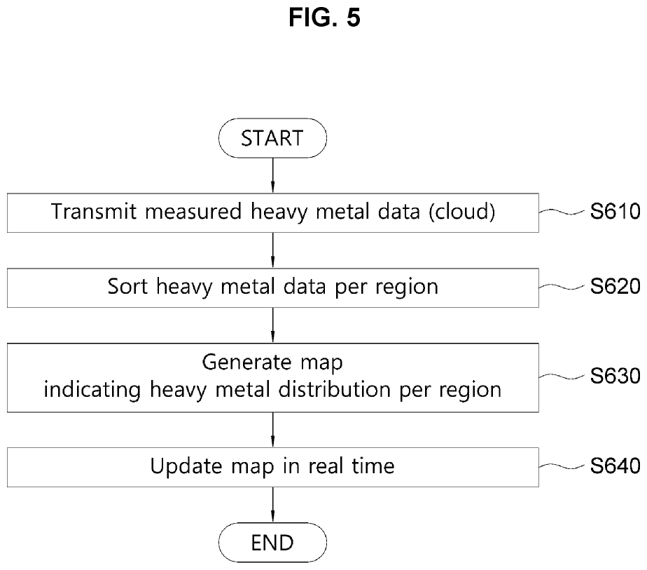

FIG. 5 is a flowchart showing a process of collecting big data, from the heavy metal measuring device (100) according to an embodiment of the present invention, and completing a platform for heavy metal measurement. As shown in the drawing, data that has been accumulated after each measurement of a heavy metal is collected and then processed as big data. Then, a tendency is extracted via deep learning, which is then transmitted to a cloud so that the extracted tendency can be operated and managed by the cloud. The extracted information is then provided to a heavy metal measurement platform, which is installed in a terminal (or user equipment), thereby allowing a platform provider or individual user to use the provided information.

That is, while collecting and analyzing data on the measured heavy metal not only from just one country but also from various regions (or locations) around the world and providing such data to the aforementioned platform, a heavy metal map may be produced (or drawn) at the same time, which may be used as an important document on the current status of heavy metal contamination. This is feasible in that the heavy metal measuring device (100) according to the present invention has been devised to be conveniently used not only by a professional user (or specialist) but also by a non-professional user regardless of the properties of the measurement object, such as its liquid state or solid state, and so on. Furthermore, due to such convenience, by allowing opportunities and frequency in heavy metal measurement to be increased, and by allowing accumulated heavy metal contamination (or pollution) information to be transmitted to a central server, such as a cloud, from any location having a communication system established therein, a precise heavy metal contamination distribution map segmented per region may be configured.

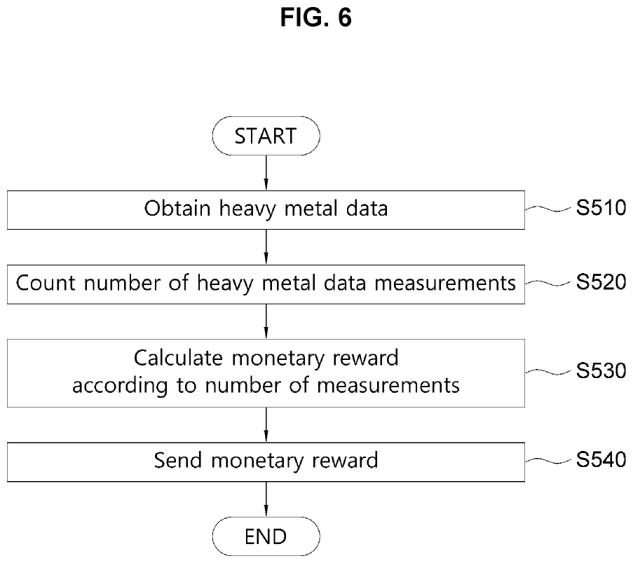

FIG. 6 is a flowchart for describing a process of collecting data by using heavy metal measuring device (100) according to an embodiment of the present invention.

The present invention includes the steps of transmitting measured heavy metal data by a wireless communication electrode, thereby storing the data in a cloud or stored the data in a cloud via a portable terminal (or user equipment); sorting the data per region; and indicating heavy metal distribution on a map based on the data being sorted per region. Herein, in case an update in the heavy metal data occurs, the heavy metal distribution is designed to be updated in real time in accordance with the updated heavy metal data.

Additionally, herein, a number of measurements of the heavy metal data measured by different measurers (or measuring people) may be counted per individual, and the counted measurements may be calculated to a monetary reward corresponding to the number of measurements, which may then be awarded (or provided) to the respective measurers. And, the calculated monetary reward may also be provided to the respective measurers on a monthly basis or a daily basis. Thus, the measurement of heavy metals may be encouraged, thereby allowing heavy metal maps to be swiftly created and updated.

That is, if the heavy metal measuring device (100) according to the present invention is distributed, for example, to residents of a particular region, and if the device is designed to provide a monetary reward for each time a heavy metal measurement is performed, the residents may actively measure heavy metals contained in various entities. And, when big data is processed from the data accumulated from various measurements, and if the processed big data is commercialized, a business program may be designed to return the profit as monetary reward to the residents who have participated in the measurement process. Accordingly, a larger number of participants may be recruited from the business program. Thus, a virtuous cycle structure for newly establishing high quality big data may be completed.

Hereinafter, an example of configuring electrodes in the form of an array, and a measurement method according to the configured array will be described in detail.

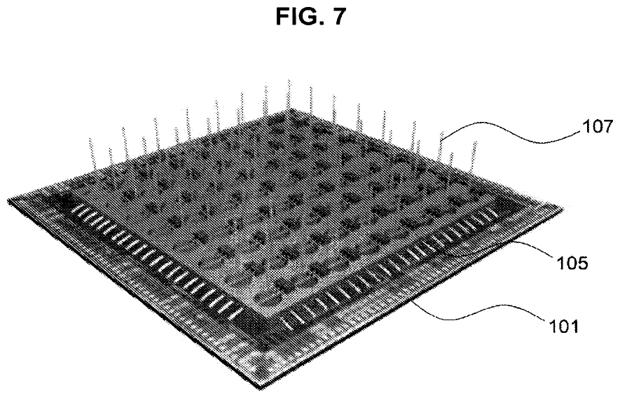

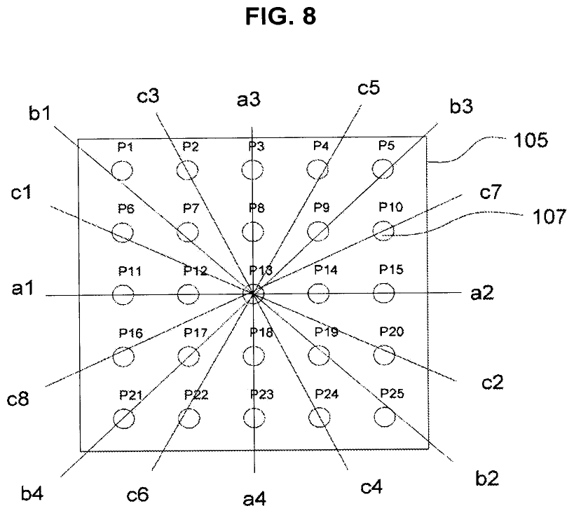

FIG. 7 is a mimetic diagram of a heavy metal measuring device (100) having an electrode part (107) array pattern according to an embodiment of the present invention. FIG. 8 to FIG. 10 show various embodiments of an electrode part (107) array pattern according to the present invention.

As shown in FIG. 7, although the array format is configured as a 3D electrode structure, the 3D-structured electrode is merely exemplary, and, therefore, a 2D-structured array electrode may also be applied.

FIG. 8 shows a mimetic diagram describing a new measurement method using an electrode part (107) having an array format proposed in the present invention. The conventional measurement method is a method of detecting heavy metals by measuring capacitance or impedance between two electrodes and by using a change in the capacitance or impedance according to the presence or absence of a heavy metal. However, as shown in the present invention, by using a large number of array-structured electrodes, by measuring various capacitance or impedance between numerous neighboring electrodes, and by observing the amount of change, the level of precision and reliability may be increased to an outstanding level.

The present invention is a heavy metal measuring device (100) having multiple electrode parts (107) aligned in a substrate (103). Herein, a reference electrode part (107), which is a reference electrode, and counter electrode groups provided with other electrode parts (107) located at different distances from the reference electrode part (107) are configured, and measurement values of capacitance or impedance formed in the reference electrode part (107) and the counter electrode groups may be obtained. Thus, measurement of the heavy metals may be performed.

The present invention may obtain an average of the measurement values per counter electrode group, and an average of the measurement values excluding at least one of a highest value or a lowest value among the measurement values per counter electrode group may also be obtained.

Generally, an average of the measurement values shall be obtained. However, in case an error is expected in the highest value or the lowest value, an average excluding such values may also be obtained.

An array pattern structure of the electrode parts (107) according to the present invention may be implemented according various embodiments.

According to an embodiment, it is preferable that multiple electrode parts (107) are formed in a pattern configured of the same number of electrode parts (107) along a longitudinal direction and a transverse direction, and that the reference electrode part (107) is any one of the multiple electrode parts (107).

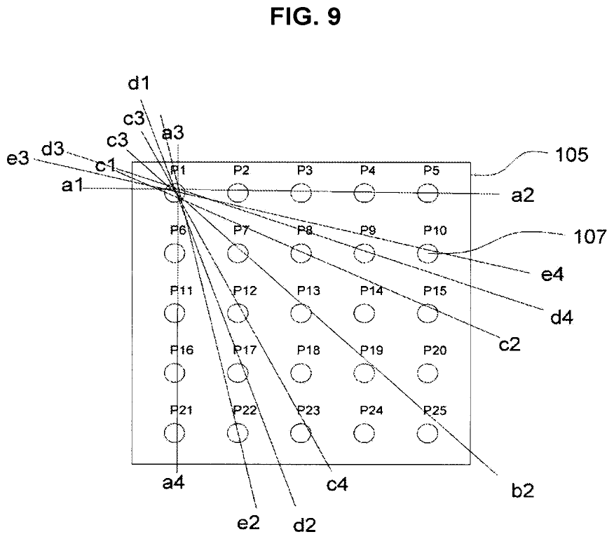

In case of the above-described embodiment, it is preferable that a same odd number of multiple electrode parts (107) are aligned along the longitudinal direction and the transverse direction (see FIG. 8 and FIG. 9), and that the reference electrode part (107) is configured as the electrode part (107) located at the center. Nevertheless, this does not mean that a case where an even number of the multiple electrode parts (107) are aligned along the longitudinal direction and the transverse direction is excluded from the scope of the claims of the present invention.

According to another embodiment, the array pattern (not shown) is formed by aligning different numbers of the multiple electrode parts (107) along the longitudinal direction and the transverse direction, and the reference electrode part (107) may be any one of the multiple electrode parts (107).

Meanwhile, in the present invention, the aforementioned ‘longitudinal direction’ and ‘transverse direction’ will not be limited only to a vertical direction and a horizontal direction, and, therefore, alignment (or positioning) along a diagonal direction may also be included. That is, there is no separate limitation in directionality.

For example, the electrode parts (107) may be freely aligned (or positioned) along any of the vertical direction, horizontal direction, diagonal direction, random direction, and so on. Herein, however, a pattern may be formed so as to be provided with a plurality of other electrode parts (107) each corresponding to the same distance from the reference electrode part (107).

According to an embodiment, FIG. 3 shows a pattern structure being configured of an array of 25 electrode parts (107). Among these electrode parts (107), if P13, which is the electrode part (107) located at the center, is configured as the reference electrode part (107), there are a total of 4 electrode parts (107) (P8, P12, P18, P14) being located at the nearest distance from the P13 electrode part (107).

In the present invention, electrode parts (107) that are located at a same distance from the reference electrode part (107) will be referred to as a counter electrode group. Accordingly, the 4 electrode parts (107) being located at a nearest distance from the reference electrode part (107) will be referred to as a first counter electrode group.

The first counter electrode group exist as the 4 electrode parts (107) (P8, P12, P18, P14) being located along line a1-a2 and line a3-a4 of FIG. 8. And, there exist 4 measurement values being measured from the reference electrode part (107).

In this case, although only one measurement value of the capacitance or impedance between two electrode parts (107) existed in the conventional method, in the present invention, since 4 nearest electrode parts (107) exist, 4 measurement values exist, accordingly.

Subsequent to the first counter electrode group, 4 electrode parts (107) (P7, P9, P17, P19) being located at a second nearest distance from the reference electrode part (107) are configured as a second counter electrode group. The second counter electrode group may exist as the 4 electrode parts (107) being located along line b1-b2 and line b3-b4 of FIG. 8. And, there exist 4 measurement values being measured from the reference electrode part (107).

Subsequent to the second counter electrode group, 4 electrode parts (107) (P3, P11, P15, P23) being located at a third nearest distance from the reference electrode part (107) are configured as a third counter electrode group. The third counter electrode group may exist as the 4 electrode parts (107) being located along line a1-a2 and line a3-a4 of FIG. 8. And, there exist 4 measurement values being measured from the reference electrode part (107).

Subsequent to the third counter electrode group, 8 electrode parts (107) (P6, P20, P2, P24, P4, P22, P10, P16) being located at a fourth nearest distance from the reference electrode part (107) are configured as a fourth counter electrode group. The fourth counter electrode group may exist as the 8 electrode parts (107) being located along line c1-c2, line c3-c4, line c5-c6, and line c7-c8 of FIG. 8. And, there exist 8 measurement values being measured from the reference electrode part (107).

Subsequent to the fourth counter electrode group, 4 electrode parts (107) (P1, P5, P21, P25) being located at a fifth nearest distance from the reference electrode part (107) are configured as a fifth counter electrode group. The fifth counter electrode group may exist as the 4 electrode parts (107) being located along line b1-b2, and line b3-b4 of FIG. 8. And, there exist 4 measurement values being measured from the reference electrode part (107).

To summarize, in case electrode P13 is configured as the reference electrode part (107), change values in capacitance or impedance between the total of 24 electrodes may be obtained, and, in comparison to the conventional measurement method, property values having much higher precision and reliability may be obtained.

FIG. 9, for example, shows an embodiment of configuring another electrode position (P1) as the reference electrode part (107). In this embodiment, by configuring counter electrode groups each corresponding to the same nearest distances and a counter electrode group corresponding to the same furthest distance, and by measuring the variance (or change values) in capacitance or impedance between the reference electrode part (107) and other electrodes, a total of 24 measurement values may be obtained.

By using this method, measurement values of the variance (or change) between a total of 25 electrode parts (107) and electrode parts (107) being adjacent to each position may be obtained, And, as described above, in case 25 electrode parts (107) exist, a total of 300 measurement values may be obtained in one measurement. Thus, precise and reliable measurement values may be obtained.

In case the number of electrode parts (107) is equal to n, the total number of measurement values that may be obtained from a single measurement may be equal to n(n−1)/2. And, therefore, in case 100 electrode parts (107) exist, a total of 4,950 measurement values may be obtained from a single measurement.

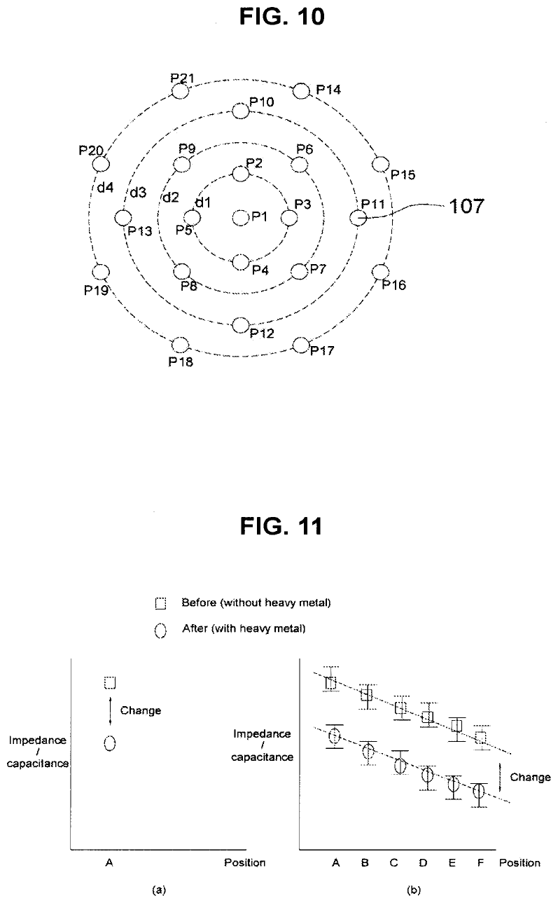

Meanwhile, FIG. 10 shows an embodiment wherein the electrode part (107) array has a pattern structure of concentric circles. As shown in FIG. 10, multiple electrode parts (107) are aligned at a center of multiple concentric circles and on the circumference of each concentric circle. Herein, the reference electrode is the electrode part (107) being located at the center of the concentric circles, and the counter electrode groups may correspond to the electrode parts (107) being aligned on the circumference of each concentric circle.

In case of FIG. 10, 4 electrode parts (107) (P2, P3, P4, P5) located at a same nearest distance (d1) from the reference electrode part (107) (P1) configure a first counter electrode group.

Subsequent to the first counter electrode group (d1), 4 electrode parts (107) (P6, P7, P8, P9) located at a same second nearest distance (d2) from the reference electrode part (107) (P1) configure a second counter electrode group.

Subsequent to the second counter electrode group (d2), 4 electrode parts (107) (P10, P11, P12, P13) located at a same third nearest distance (d3) from the reference electrode part (107) (P1) configure a third counter electrode group.

Subsequent to the third counter electrode group (d3), 8 electrode parts (107) (P14, P15, P16, P17, P18, P19, P20, P21) located at a same fourth nearest distance (d4) from the reference electrode part (107) (P1) configure a fourth counter electrode group.

As proposed in the various embodiments, which are described above, the present invention may indicate a distribution map of numerous measurement values, and a more precise comparison of the variance between the electrodes according to the measurement object heavy metals may be carried out. Thus, precise heavy metal detection may be performed.

FIG. 11 shows comparison data in results from measuring properties by using the method proposed in the present invention. FIG. 11a shows data being measured between two electrodes by using the conventional method. FIG. 11b shows data being measured by using the electrode array and the simultaneous measurement method, which are proposed in the present invention.

Referring to FIG. 11, red rectangular markings indicate impedance or capacitance measurement values between electrodes in a reference state where heavy metal does not exist (i.e., a state without heavy metal). Also, referring to FIG. 11, blue circular markings are measurement values corresponding to a state where heavy metal exists (i.e., a state with heavy metal). And, the presence or absence of heavy metal may be predicted based on the variance (or change) between the two states.

According to the conventional method shown in FIG. 11a, since only two electrodes exist, only one measurement data between the electrodes exists. That is, since there is only one measurement value in the reference state, and since there is only one changed (or varied) measurement value, only one constant data value may be obtained as the variance.

However, in an actual state, since the forms of the electrodes are not completely consistent, there are limitations in making an accurate diagnosis based on only one data variation value.

However, since the present invention uses a multi-electrode array, and since multiple electrode parts (107) (first counter electrode group) are located at a nearest distance from a single reference electrode part (107), more reliable measurement values may be obtained from multiple average values.

Additionally, since an electrode array is used, as shown in the examples of FIG. 7 to FIG. 10, a plurality of electrode part (107) (counter electrode group) positions may be located at different sets of equal distances from the reference electrode part (107). Therefore, as shown in FIG. 11b, an average measurement value of various counter electrode groups may be obtained from various positions (positions B, C, D, E, F) at the same distance. Thus, since the parameters for performing comparison with a reference state are increased, an accurate diagnosis may be made by performing a more precise comparison.

Moreover, since the variance of the impedance or capacitance according to each position may be indicated in a graph, the variance (or change) may be indicated by a change in inclinations in the graph, and not by a simple change (or variance) in the constant.

FIG. 11 shows an example wherein the impedance or capacitance measurement value decreases in the state with heavy metal as compared to the reference state. However, depending upon the type of heavy metal, such measurement value may be increased. And, in such case, the comparison of variance (or change) may also be equally applied as described above.

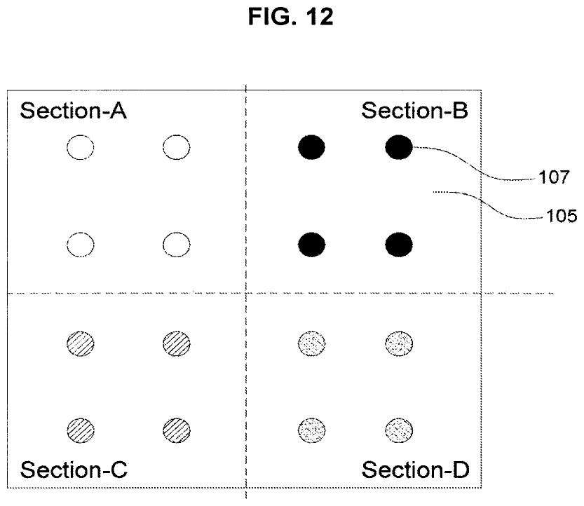

FIG. 12 shows an embodiment of an array pattern being divided into multiple sections according to an embodiment of the present invention. More specifically, FIG. 12 shows a mimetic diagram showing a method for detecting multiple heavy metals at once, instead of only one heavy metal, in a single electrode array structure.

As described above, depending upon the type of heavy metal, the impedance or capacitance measurement value may be varied. And, in this case, the electrode array may be divided into multiple arbitrary sections. And, by adjusting the electrode structure or the receptor (109) attached to the electrodes so that different heavy metals can be detected from each divided section, various heavy metals may be simultaneously detected at once.

In FIG. 12, as a random embodiment, an electrode array structure is divided into 4 different sections (section-A, B, C, D), and each section may be configured to detect a different heavy metal.

In FIG. 12, for example, if section-A is configured to detect Mercury, if section-B is configured to detect Lead, if section-C is configured to detect Cadmium, and if section-D is configured to detect Arsenic, the various types of heavy metals may all be detected at once.

Additionally, in case of the embodiment of FIG. 12, each section is configured of 4 electrode parts (107). Therefore, as described above in the embodiments of FIG. 8 and FIG. 9, six measurement data may be obtained for each section. Thus, even if multiple heavy metals are being detected and measured, high levels of precision and reliability may be ensured.

As described above, the heavy metal measuring device (100) according to the present invention may obtain highly precise and reliable data. Moreover, a heavy metal measuring device (100) and a measurement method using the same that can simultaneously detect various heavy metals from a single measurement may be implemented.

It shall be noted that the embodiments set forth herein are provided to describe the embodiments according to the present invention, and not to limit the present invention. Furthermore, it may be understood by anyone with ordinary skills in the field that other various embodiments may also be implemented without deviating from the technical scope and spirit of the present invention.

Description of reference numerals

| | | | |
|---|---|---|---|
| 100: | measuring device | 101: | controller |
| 103: | substrate | 105: | base substrate |
| 107: | electrode part | 109: | receptor |

What is claimed is:

1. A device for measuring heavy metals having an electrode element, the device comprising:
a substrate having a wireless communication electrode for wirelessly delivering an electrical variance that is measured from an electrode, when measuring heavy metals;
an electrode element delivering an electrical signal for measuring heavy metals; and
a controller controlling electric current application to the electrode element and detecting a quantity of electricity between electrodes being measured from a measurement object contaminated by heavy metal, or calculating a displacement between a reference quantity of electricity and a measured quantity of electricity.

2. The device of claim 1, wherein the electrode element comprises:
a base substrate; and
at least one or more pillar-type electrode parts protruding from the base substrate, or a flat-type electrode part.

3. The device of claim 2, wherein the electrode part is coated with a receptor reacting to each heavy metal.

4. The device of claim 2, wherein the electrode part is configured of multiple microelectrodes,
wherein counter electrode groups are configured of one microelectrode being a reference electrode and other microelectrodes each being positioned at different positions at a same distance from the reference electrode, and
wherein capacitance or impedance measurement values are obtained between the reference electrode and each microelectrode of the counter electrode groups.

5. The device of claim 4, wherein an average value of measurement values for each counter electrode group is obtained, or an average value of the measurement values excluding at least one of a highest value or a lowest value, among the measurement values for each counter electrode group, is obtained.

6. The device of claim 4, wherein multiple microelectrodes configure a pattern of a same number of microelectrodes or different numbers of microelectrodes being aligned along a longitudinal direction and a transverse direction.

7. The device of claim 4, wherein multiple microelectrodes are positioned at a center of multiple concentric circles and aligned along a circumference of each concentric circle, wherein a reference electrode is a microelectrode being positioned at the center, and wherein the counter electrode group is a group of microelectrodes being aligned along the circumference of each concentric circle.

8. The device of claim 4, wherein multiple microelectrodes being aligned on the substrate are divided into a predetermined plurality of sections, and
wherein the microelectrodes of each section are configured to detect substances that are different from one another.

9. A method for measuring heavy metals by the measuring device of claim 1 and performing update in real time, the method comprising:
transmitting measured heavy metal data by a wireless communication electrode, thereby allowing heavy metal data to be stored in a cloud or allowing heavy metal data to be stored in a cloud via a portable terminal;
sorting the data per region; and
marking heavy metal distribution on a map based on the data being sorted per region,
wherein the heavy metal distribution is updated in real time when updating the heavy metal data.

10. The method of claim 9, comprising:
counting a number of measurements of the heavy metal data;
performing calculation to convert the measurement numbers to a monetary reward for each measurer; and
providing the monetary reward to each measurer.
